(19) United States
(12) Patent Application Publication
(10) Pub. No.: US 2019/0358135 A1
(43) Pub. Date: Nov. 28, 2019

(54) COSMETIC COMPOSITION

(71) Applicant: OTSUKA CHEMICAL CO., LTD., Osaka-city, Osaka (JP)

(72) Inventors: Kosuke Inada, Tokyo (JP); Haruna Nishimoto, Osaka-city (JP); Takashi Hamauzu, Tokushima-city (JP); Hiroyoshi Mori, Tokushima-city (JP)

(73) Assignee: OTSUKA CHEMICAL CO., LTD., Osaka-city, Osaka (JP)

(21) Appl. No.: 16/479,376

(22) PCT Filed: Feb. 5, 2018
(86) PCT No.: PCT/JP2018/003717

§ 371 Date: Jul. 19, 2019

(30) Foreign Application Priority Data
Feb. 7, 2017 (JP) 2017-020119
Mar. 22, 2017 (JP) 2017-055287

(51) Int. Cl. A61K 8/29; A61K 8/25; A61K 8/26; A61K 8/27; A61K 8/28; A61K 8/02

(52) U.S. Cl. CPC A61K 8/29; A61K 8/25; A61K 8/26; A61K 8/27; A61Q 17/04; A61K 8/0254; A61K 2800/43; A61K 2800/412; A61K 8/28

(57) ABSTRACT

Provided is a cosmetic composition containing metal oxide particles having improved dispersibility. A cosmetic composition contains metal oxide particles with an average particle diameter of 1 μm or less and lepidocrocite-type platy titanate particles with an average unrolled diameter of 0.1 μm to 10.0 μm and an average thickness of 0.1 μm to 4.0 μm and the titanate particles are at least one selected from titanates expressed by chemical formulae K_{0.5-0.7}Li_{0.27}Ti_{1.73}O_{3.85-3.95}, K_{0.2-0.7}Mg_{0.4}Ti_{1.6}O_{3.7-3.95}, and K_{0.2-0.7}Li_{0.27-(2x/3)}MgₓTi_{1.73-(x/3)}O_{3.7-3.95} [where 0.004≤x≤0.4].

### TECHNICAL FIELD

The present invention relates to cosmetic compositions containing metal oxide particles.

### BACKGROUND ART

Generally, metal oxide particles with a particle diameter of about 0.01 μm to 1 μm are used as color pigments, white pigments, and extender pigments for cosmetic materials. The light scattering power of such particulate powder is a function of its particle diameter and light wavelength. For example, regarding titanium dioxide, the scattering power for visible light reaches a maximum when the particle diameter is within a range of 0.2 μm to 0.3 am, in which case the titanium dioxide powder can hide a base and achieve a high degree of whiteness. On the other hand, when the particle diameter is smaller than the range of 0.2 μm to 0.3 μm, this means departure from the particle diameter range within which the hiding power reaches a maximum, thus reducing the scattering power for visible light to provide transparency and, concurrently, the ultraviolet blocking properties increase. By taking advantage of these kinds of properties, titanium dioxide with a particle diameter of 0.2 μm to 0.3 μm is used for cosmetic materials for makeup or the like and titanium dioxide with a particle diameter of 0.1 μm or less is used for sunscreen cosmetic materials or the like. However, as the particle size decreases, the interparticle cohesion increases and agglomerated particles (secondary particles) are more difficult to disperse, which prevents sufficient exertion of capabilities of the particles.

Therefore, the particles are used by bringing them close to the form of primary particles by mechanical dispersion, but this is still insufficient to achieve the sufficient capabilities. As a solution to this, Patent Literature 1 proposes to use a dispersion medium and a dispersant. Patent Literature 2 proposes to use as a base material platy α-alumina particles with an average particle diameter of 0.5 μm to 20 μm, an average thickness of 0.03 μm to 0.35 μm, and an aspect ratio of 15 to 50 and fix 30 to 50% by mass titanium dioxide to the surfaces of the particles.

Meanwhile, Patent Literature 3 proposes a luster pigment which is lepidocrocite-type, platy crystalline titanate particles having an average thickness of 0.1 μm to 5 μm and an average length of 10 μm to 100 μm and selected from the group consisting of chemical formulae K₃ₓLiₓTi₂₋ₓO₄, K₂ₓMgₓTi₂₋ₓO₄, and KₓFeₓTi₂₋ₓO₄ (in all of which 0.05≤x≤0.5).

### CITATION LIST

### Patent Literature

Patent Literature 1: JP-A-H06-239728

Patent Literature 2: JP-A-2008-88317

Patent Literature 3: JP-A-2008-162971

### SUMMARY OF INVENTION

### Technical Problem

Cosmetic compositions directly touch someone's skin. Therefore, using substances not directly involved in the functions of the cosmetic compositions should preferably be avoided. However, the method disclosed in Patent Literature 1 uses a dispersant not directly involved in the functions of cosmetic compositions.

In the method disclosed in Patent Literature 2, since the base material particles have a large particle diameter, the method cannot be expected to increase the ultraviolet blocking properties which particulate titanium dioxide has. Also for other types of metal oxide particles, there is concern that the same problem will occur as with titanium dioxide.

The present invention has been made in view of the above circumstances and therefore has a principal object of providing a cosmetic composition containing metal oxide particles having improved dispersibility.

### Solution to Problem

The inventors found that the use of metal oxide particles in combination with specific platy titanate particles increases the dispersibility of the metal oxide particles and completed the present invention.

Aspect 1: A cosmetic composition containing metal oxide particles with an average particle diameter of 1 μm or less and lepidocrocite-type platy titanate particles with an average unrolled diameter of 0.1 μm to 10.0 μm and an average thickness of 0.1 μm to 4.0 μm, the titanate particles being at least one selected from titanates expressed by chemical formulae K_{0.5-0.7}Li_{0.27}Ti_{1.73}O_{3.85-3.95}, K_{0.2-0.7}Mg_{0.4}Ti_{1.6}O_{3.7-3.95}, and K_{0.2-0.7}Li_{0.27-(2x/3)}MgₓTi_{1.73-(x/3)}O_{3.7-3.95} [where 0.004≤x≤0.4].

Aspect 2: The cosmetic composition according to aspect 1, wherein the metal oxide particles are at least one selected from the group consisting of titanium dioxide, zinc oxide, iron oxide, aluminum oxide, cerium oxide, zirconium oxide, silicon oxide, chromium oxide, magnesium oxide, and black titanium oxide.

Aspect 3: The cosmetic composition according to aspect 1 or 2, wherein the metal oxide particles have an average particle diameter of 0.01 μm to 0.5 μm.

Aspect 4: The cosmetic composition according to any one of aspects 1 to 3, wherein the titanate particles have an average length of less than 10 μm.

Aspect 5: The cosmetic composition according to any one of aspects 1 to 4, wherein the titanate particles have an average unrolled diameter ratio of 1 to 5.

Aspect 6: The cosmetic composition according to any one of aspects 1 to 5, wherein a content of the titanate particles is 0.1 to 200 parts by mass relative to 100 parts by mass of the metal oxide particles.

### Advantageous Effects of Invention

According to the present invention, the use of metal oxide particles in combination with specific platy titanate particles increases the dispersibility of the metal oxide particles and thus enables sufficient exertion of capabilities that the metal oxide particles have, so that, for example, when titanium dioxide is selected as the metal oxide particles, a cosmetic composition having excellent hideability and ultraviolet blocking properties can be provided.

### DESCRIPTION OF EMBODIMENTS

Hereinafter, a description will be given of preferred embodiments. However, the following embodiments are simply illustrative and the present invention is not intended to be limited to the following embodiments.

A cosmetic composition according to the present invention contains metal oxide particles with an average particle diameter of 1 μm or less and lepidocrocite-type platy titanate particles with an average unrolled diameter of 0.1 μm to 10.0 μm and an average thickness of 0.1 μm to 4.0 μm, and the titanate particles are at least one selected from titanates expressed by chemical formulae K_{0.5-0.7}Li_{0.27}Ti_{1.73}O_{3.85-3.95}, K_{0.2-0.7}Mg_{0.4}Ti_{1.6}O_{3.7-3.95}, and K_{0.2-0.7}Li_{0.27-(2x/3)}MgₓTi_{1.73-(x/3)}O_{3.7-3.95} [where 0.004≤x≤0.4]. Furthermore, the cosmetic composition according to the present invention may contain, in addition to metal oxide particles and lepidocrocite-type platy titanate particles to be described hereinafter, other components as necessary.

A description will be given below of each of the components of the cosmetic composition according to the present invention.

<Metal Oxide Particles>

The metal oxide particles for use in the present invention have an average particle diameter of 1 μm or less and preferably have an average particle diameter of 0.01 μm to 0.5 μm. Note that the average particle diameter in the present invention refers to a median diameter of primary particles measured by electron microscopy.

So long as the metal oxide particles are those commonly used as a cosmetic composition, there is no limitation as to their particle shape, such as spherical, their particle structure, such as porous or non-porous, and other characteristics. Specific examples include titanium dioxide, zinc oxide, iron oxide, aluminum oxide, cerium oxide, zirconium oxide, silicon oxide, chromium oxide, magnesium oxide, and black titanium oxide. Examples also include composite powders containing these kinds of metal oxide particles and these kinds of metal oxide particles can be used singly or in combination of two or more thereof. If necessary, the metal oxide particles may be subjected to surface treatment by any known method using, for example, a silicone-based compound, a fluorine-based compound, metallic soap, collagen, hydrocarbon, higher fatty acid, higher alcohol, ester, wax or a surfactant.

The metal oxide particles for use in the present invention can be used as a white pigment, a red pigment, a yellow pigment, a black pigment, a luster pigment, an ultraviolet blocker or so on according to the kind of metal oxide particles selected.

For example, regarding titanium dioxide particles, the scattering power for visible light reaches a maximum when the particle diameter is within a range of 0.2 μm to 0.3 μm, in which case titanium dioxide particles can hide a base and achieve a high degree of whiteness. Therefore, titanium dioxide particles with an average particle diameter of 0.1 μm to 0.5 μm can be suitably used as a white pigment.

On the other hand, when the particle diameter is smaller than the range of 0.2 μm to 0.3 μm, this means departure from the particle diameter range within which the hiding power reaches a maximum, thus reducing the scattering power for visible light to provide transparency and, concurrently, the ultraviolet blocking properties increase. Therefore, titanium dioxide particles with an average particle diameter of 0.01 μm to 0.07 m can be suitably used as an ultraviolet blocker for sunscreen cosmetics. There are three types of crystal structures of titanium dioxide particles: rutile, brookite, and anatase, but any one of them may be used.

Regarding zinc oxide particles, those having an average particle diameter of 0.3 μm to 0.7 μm and preferably 0.3 μm to 0.5 μm can be mixed as a white pigment. Furthermore, because zinc oxide particles have a weak astringent action on skin, they can be mixed into cosmetics for soothing the redness of sun-damaged skin or the like.

Colors of iron oxide particles include red, yellow, and black depending on the degree of oxidation of iron. For example, colcothar containing ferric oxide (Fe₂O₃) as a main ingredient can be used as a red pigment. Furthermore, as for iron oxide particles, those having an average particle diameter of 0.1 μm to 0.5 μm can be suitably used.

<Titanate Particles>

The titanate particles for use in the present invention are lepidocrocite-type titanate particles with an average unrolled diameter of 0.1 μm to 10.0 μm and an average thickness of 0.1 μm to 4.0 μm. Furthermore, the shape of the titanate particles for use in the present invention is platy.

The average unrolled diameter of the titanate particles for use in the present invention is preferably 0.1 μm to 8.0 μm and more preferably 0.5 μm to 5.0 μm. The average thickness thereof is preferably 0.1 μm to 2.0 μm and more preferably 0.1 m to 1.5 μm. The average length thereof is preferably less than 10 μm, more preferably 0.1 μm to 4.0 μm, and still more preferably 0.5 μm to 4.0 μm. The average unrolled diameter ratio is preferably 1 to 5 and more preferably 1 to 3. When the average unrolled diameter, the average thickness, the average length, and the average unrolled diameter ratio are within the respective ranges described above, the use of titanate particle in combination with the metal oxide particles enables further prevention of agglomeration of the metal oxide particles and thus further improvement in the dispersibility of metal oxide particles.

The average length, average unrolled diameter, and average unrolled diameter ratio of the titanate particles were obtained in the following manners. First, any 50 particles were selected by scanning electron microscopic (SEM) observation and their lengths and breadths were measured. The average length was obtained from the arithmetic average of the lengths of the 50 particles. The average unrolled diameter was obtained from the arithmetic average of the values of ((length)+(breadth))/2 of the 50 particles. The average unrolled diameter ratio was obtained from the arithmetic average of the values of (length)/(breadth) of the 50 particles. Furthermore, the average thickness of the titanate particles was obtained by selecting any 10 particles by SEM observation, measuring their thicknesses, and taking the arithmetic average of the thicknesses of the 10 particles.

The titanate particles for use in the present invention are selected from titanates expressed by chemical formulae K_{0.5-0.7}Li_{0.27}Ti_{1.73}O_{3.85-3.95}, K_{0.2-0.7}Mg_{0.4}Ti_{1.6}O_{3.7-3.95}, and K_{0.2-0.7}Li_{0.27-(2x/3)}MgₓTi_{1.73-(x/3)}O_{3.7-3.95} [where 0.004≤x≤0.4], preferably selected from titanates expressed by chemical formulae K_{0.5-0.7}Li_{0.27}Ti_{1.73}O_{3.85-3.95}, K_{0.5-0.7}Mg_{0.4}Ti_{1.6}O_{3.85-3.95}, and K_{0.5-0.7}Li_{0.27-(2x/3)}MgₓTi_{1.73-(x/3)}O_{3.85-3.95} [where 0.004≤x≤0.2], and is more preferably K_{0.5-0.7}Li_{0.27-(2x/3)}MgₓTi_{1.73-(x/3)}O_{3.85-3.95} [where 0.004≤x≤0.2] from the viewpoint of further preventing elution of inter-layer potassium ions. These kinds of titanate particles can be used singly or in combination of two or more thereof. The above compositions each have an orthorhombic layered structure and have a platy shape as with mica or the like, but have a feature that, as compared to mica or the like, the gloss and luster do not change greatly even when viewed from different angles (i.e., the angular dependency is small).

The method for producing the above K_{0.5-0.7}Li_{0.27}Ti_{1.73}O_{3.85-3.95} is, for example, as disclosed in WO 2003/037797. The method for producing K_{0.2-0.7}Mg_{0.4}Ti_{1.6}O_{3.7-3.95} is, for example, as disclosed in WO 2002/010069. The method for producing K_{0.2-0.7}Li_{0.27-(2x/3)}MgₓTi_{1.73-(x/3)}O_{3.7-3.95} [where 0.004≤x≤0.4] is, for example, as disclosed in WO 2015/045954. Specifically, the above compositions can be obtained by preparing as raw materials a compound forming titanium dioxide by application of heat or titanium dioxide (a titanium source), a compound forming potassium oxide by application of heat or potassium oxide (a potassium source), if necessary, a compound forming lithium oxide by application of heat or lithium oxide (a lithium source), and, if necessary, a compound forming magnesium oxide by application of heat or magnesium oxide (a magnesium source), mixing these materials together, if necessary, with the addition of a flux for the purposes of reaction homogenization and/or crystal growth, firing (primarily firing) the obtained raw material mixture, eluting potassium from the obtained primarily fired product, then drying the primarily fired product, and, if necessary, firing (secondarily firing) the primarily fired product. The surface treatment for the titanate particles is made still easier by avoiding the secondary firing, but conducting the secondary firing is preferred from the viewpoint of further increasing the stability of crystals of the titanate particles.

No particular limitation is placed on the titanium source so long as it is a raw material (a compound) containing titanium elements and not inhibiting the formation of titanium dioxide by application of heat or titanium dioxide, but examples of the compound include titanium dioxide, titanium suboxide, orthotitanic acid, salts of orthotitanic acid, metatitanic acid, salts of metatitanic acid, titanium hydroxide, peroxotitanic acid, and salts of peroxotitanic acid. These compounds can be used singly or in combination of two or more thereof. Preferred among them is titanium dioxide. The crystal shape of titanium dioxide is preferably rutile or anatase.

No particular limitation is placed on the potassium source so long as it is a raw material (a compound) containing potassium elements and not inhibiting the formation of potassium oxide by application of heat or potassium oxide, but examples of the compound include potassium oxide, potassium carbonate, and potassium hydroxide. These compounds can be used singly or in combination of two or more thereof. Preferred among them is potassium carbonate.

No particular limitation is placed on the lithium source so long as it is a raw material (a compound) containing lithium elements and not inhibiting the formation of lithium oxide by application of heat or lithium oxide, but examples of the compound include lithium oxide, lithium hydroxide, lithium carbonate, and lithium fluoride. These compounds can be used singly or in combination of two or more thereof. Preferred among them is lithium carbonate.

No particular limitation is placed on the magnesium source so long as it is a raw material (a compound) containing magnesium elements and not inhibiting the formation of magnesium oxide by application of heat or magnesium oxide, but examples of the compound include magnesium hydroxide, magnesium carbonate, and magnesium fluoride. These compounds can be used singly or in combination of two or more thereof. Preferred among them is magnesium hydroxide.

For example, in the case of K_{0.7}Li_{0.27}Ti_{1.73}O_{3.95}, the mixing ratio of the titanium source, the potassium source, and the lithium source is basically Ti:K:Li=1.73:0.8:0.27 (by molar ratio), but a change of about 5% in the content of each source will present no problem. Large departure from the above ratio may cause precipitation of non-platy side products, Li₂TiO₃, K₂Ti₆O₁₃, and K₂Ti₄O₉ and is therefore not preferred.

In the case of K_{0.7}Mg_{0.4}Ti_{1.6}O_{3.95}, the mixing ratio of the titanium source, the potassium source, and the magnesium source is basically Ti:K:Mg=1.6:0.8:0.4 (by molar ratio), but a change of about 5% in the content of each source will present no problem. Large departure from the above composition may cause precipitation of non-platy side products, MgₓTiO₃, K₂Ti₆O₁₃, and K₂Ti₄O₉ and is therefore not preferred. In the case of K_{0.7}Li_{0.14}Mg_{0.2}Ti_{1.66}O_{3.95}, the mixing ratio of the titanium source, the potassium source, the lithium source, and the magnesium source is basically Ti:K:Li:Mg=1.66:0.8:0.14:0.2 (by molar ratio), but a change of about 5% in the content of each source will present no problem. Large departure from the above ratio may cause precipitation of non-platy side products, Li₂TiO₃, MgₓTiO₃, K₂Ti₆O₁₃, and K₂Ti₄O₉ and is therefore not preferred.

Examples of the flux that can be cited include potassium chloride, potassium fluoride, potassium molybdate, and potassium tungstate. Preferred among them is potassium chloride. The mixing ratio of the flux is preferably 10 to 100 parts by mass and more preferably 40 to 80 parts by mass, relative to 100 parts by mass of the above raw materials (the total amount of the titanium source, the potassium source, the lithium source and the magnesium source). Limiting the mixing ratio of the flux within the above range is preferred because the number of asperities formed on the particle surfaces is small and the angular dependency is made even smaller.

The primary firing is performed using an electric furnace, a rotary kiln, a tubular furnace, a fluidized firing furnace, a tunnel kiln or the like and the firing reaction can be completed by holding the raw material mixture within a temperature range of 800 to 1150° C. for 1 to 24 hours.

The elution of potassium can be performed by mixing an acid into an aqueous slurry of the primarily fired product to adjust the pH of the aqueous slurry. There is no particular limitation as to the concentration of the aqueous slurry and it can be appropriately selected from a wide range of concentrations, but, in view of the workability and so on, it is, for example, about 1 to 30% by mass and preferably about 2 to 20% by mass. Examples of the acid that can be cited include inorganic acids, such as sulfuric acid, hydrochloric acid, and nitric acid, and organic acids, such as acetic acid. The acid may be used in combination of two or more kinds of acids as necessary.

The amount of acid added into the aqueous slurry is such an amount that the pH of the aqueous slurry preferably falls within a range of 7 to 11 and more preferably falls within a range of 7 to 9. The measurement of the pH of the aqueous slurry is made after the addition of the acid into the aqueous slurry and the stirring of the mixture for about one to five hours. The acid is normally used in the form of an aqueous solution. There is no particular limitation as to the concentration of the acid aqueous solution and it can be appropriately selected within a wide range of concentrations, but it is normally about 1 to 98% by mass. After the pH of the aqueous slurry is adjusted within the above specified range, the solid content is separated from the slurry by filtration, centrifugation or other processes. The separated solid content may be, if necessary, washed with water and dried.

The secondary firing is performed using an electric furnace, a rotary kiln, a tubular furnace, a fluidized firing furnace, a tunnel kiln or so on and the firing reaction can be completed by holding the solid content obtained by the elution of potassium within a temperature range of 400 to 700° C. for 1 to 24 hours. After the secondary firing, the resultant powder may be ground into a desired size or passed through a sieve to loosen it.

For example, in the above manner, the titanate particles according to the present invention can be obtained.

If necessary, the titanate particles according to the present invention may be subjected to surface treatment by any known method using, for example, a silicone-based compound, a fluorine-based compound, metallic soap, collagen, hydrocarbon, higher fatty acid, higher alcohol, ester, wax or a surfactant.

<Other Components>

The cosmetic composition according to the present invention may contain optional components (other components) that may be added to cosmetic compositions, without impairing the effects of the present invention.

Examples of the other components include water, deionized water, oil and fat, hydrocarbon, higher fatty acid, higher alcohol, silicone, anionic surfactant, cationic surfactant, amphoteric surfactant, non-ionic surfactant, antiseptic, sequestrant, polymer compound, thickener, powder component, ultraviolet absorber, ultraviolet blocker, moisturizer, and medicinal component.

Examples of the oil and fat include: liquid oils, such as camellia oil, evening primrose oil, macadamia nut oil, olive oil, rapeseed oil, cone oil, sesame oil, jojoba oil, germ oil, wheat germ oil, and glycerin trioctanoate; solid oils and fats, such as cacao butter, coconut oil, hydrogenated coconut oil, palm oil, palm kernel oil, wood wax, wood wax kernel oil, hydrogenated oil, and hydrogenated castor oil; and waxes, such as beeswax, candelilla wax, cotton wax, bran wax, lanolin, lanolin acetate, liquid lanolin, and sugar cane wax.

Examples of the hydrocarbon include petrolatum, liquid paraffin, squalene, squalane, and microcrystalline wax.

Examples of the higher fatty acid include lauric acid, myristic acid, palmitic acid, stearic acid, oleic acid, linoleic acid, linolenic acid, docosahexaenoic acid (DHA), and eicosapentaenoic acid (EPA).

Examples of the higher alcohol include: straight-chain alcohols, such as lauryl alcohol, stearyl alcohol, cetyl alcohol, and cetostearyl alcohol; and branched-chain alcohols, such as glycerin monostearyl ether, lanolin alcohol, cholesterol, phytosterol, and octyldodecanol.

Examples of the silicone include: chain polysiloxanes, such as dimethylpolysiloxane and methylphenylpolysiloxane; and cyclic polysiloxanes, such as decamethylcyclopentasiloxane and cyclopentasiloxane.

Examples of the anionic surfactant include: fatty acid salts, such as sodium laurate; higher alkyl sulfates, such as sodium lauryl sulfate; alkyl ether sulfates, such as POE-triethanolamine lauryl sulfate; N-acyl sarcosine acid; sulfosuccinates; and N-acyl amino acid salts.

Examples of the cationic surfactant include: alkyltrimethylammonium salts, such as stearyltrimethylammonium chloride; benzalkonium chloride; and benzethonium chloride.

Examples of the amphoteric surfactant include betaine surfactants, such as alkyl betaine and amido betaine.

Examples of the non-ionic surfactant include: sorbitan fatty acid esters, such as sorbitan monooleate; and hydrogenated castor oil derivatives.

Examples of the antiseptic include methyl paraben and ethyl paraben.

Examples of the sequestrant include: disodium ethylenediaminetetraacetate; edetic acid; and edetates, such as edetic acid sodium salt.

Examples of the polymer compound include gum arabic, tragacanth gum, galactan, guar gum, carrageenan, pectin, agar, quince seed, dextran, pullulan, carboxymethyl starch, collagen, casein, gelatin, methylcellulose, hydroxypropyl methylcellulose, hydroxyethylcellulose, carboxymethylcellulose sodium (CMC), sodium alginate, and carboxy vinyl polymer (CARBOPOL).

Examples of the thickener include carrageenan, tragacanth gum, quince seed, casein, dextrin, gelatin, CMC, hydroxyethylcellulose, hydroxypropylcellulose, guar gum, xanthan gum, and bentonite.

The powder component is other than the above-described metal oxide particles and titanate particles and examples include: inorganic white pigments, such as barium sulfate; colored inorganic pigments, such as carbon black; white extender powders, such as talc, white mica, brown mica, lepidolite, black mica, synthetic mica, sericite, synthetic sericite, spherical silicone powder, silicon carbide, diatom earth, aluminum silicate, aluminum magnesium metasilicate, calcium silicate, barium silicate, magnesium silicate, calcium carbonate, magnesium carbonate, hydroxyapatite, and boron nitride; clay minerals, such as kaolin, bentonite, smectite, hectorite, and montmorillonite, and their organic modified products; glittering powders, such as titanium dioxide-coated mica, titanium dioxide-coated bismuth oxychloride, iron oxide-coated titanated mica, iron blue-treated titanated mica, carmine-treated titanated mica, bismuth oxychloride, argentine, polyethylene terephthalate-aluminum-epoxy layered powder, polyethylene terephthalate-polyolefin layered film powder, polyethylene terephthalate-polymethyl methacrylate layered film powder, and titanium oxide-coated glass flakes; organic polymeric resin powders, such as polyamide resins, polyethylene resins, polyacrylic resins, polyester resins, fluorine resins, cellulose resins, polystyrene resins, styrene-acrylic copolymer resins, polypropylene resins, silicone resins, and urethane resins; low molecular weight organic powders, such as zinc stearate and N-acyl lysine; natural organic powders, such as silk powder and cellulose powder; organic pigment powders, such as Red No. 201, Red No. 202, Red No. 205, Red No. 226, Red No. 228, Orange No. 203, Orange No. 204, Blue No. 404, and Yellow No. 401; and metal powders, such as aluminum powder, gold powder, and silver powder.

Examples of the ultraviolet absorber include para-aminobenzoic acid, phenyl salicylate, isopropyl para-methoxycinnamate, octyl para-methoxycinnamate, and 2,4-dihydroxybenzophenone.

Examples of the ultraviolet blocker include talc, carmine, bentonite, and kaolin.

Examples of the moisturizer include diisostearyl malate, polyethylene glycol, propylene glycol, dipropylene glycol, 1,3-butylene glycol, 1,2-pentanediol, glycerin, diglycerin, polyglycerin, xylitol, maltitol, maltose, sorbitol, glucose, fructose, chondroitin sulfate sodium, sodium hyaluronate, sodium lactate, pyrrolidone carboxylic acid, and cyclodextrin.

Examples of the medicinal component include: vitamin A compounds, such as vitamin A oil and retinol; vitamin B2 compounds, such as riboflavin; B6 compounds, such as pyridoxine hydrochloride; vitamin C compounds, such as L-ascorbic acid, L-ascorbic acid phosphate ester, L-ascorbic acid monopalmitate ester, L-ascorbic acid dipalmitate ester, and L-ascorbic acid-2-glucoside; pantothenates, such as calcium pantothenate; vitamin D compounds, such as vitamin D2 and cholecalciferol; vitamin E compounds, such as α-tocopherol, tocopherol acetate, and dl-α-tocopherol nicotinate; skin-lightening agents, such as placenta extract, glutathione, and saxifrage extra; skin activators, such as royal jelly and beech tree extract; blood circulation promoters, such as capsaicin, zingerone, cantharis tincture, ichthammol, caffeine, tannic acid, and γ-orizanol; antiphlogistics, such as glycyrrhizinic acid derivatives, glycyrrhetinic derivatives, and azulene; amino acids, such as arginine, serine, leucine, and tryptophan; maltose-sucrose condensate as a normal flora controlling agent; lysozyme chloride; and various extracts, such as Chamomilla recutita extract, parsley extract, wine yeast extract, grapefruit extract, Lonicera japonica extract, rice extract, grape extract, hop extract, rice bran extract, loquat extract, phellodendron bark extract, coix extract, swertia herb extract, sweet clover extract, birch extract, glycyrrhiza extract, peony root extract, saponaria extract, Luffa cylindrica extract, capsicum extract, Citrus limon fruit extract, Gentiana lutea extract, perilla extract, aloe extract, rosemary extract, sage extract, thyme extract, green tea extract, seaweed extract, cucumber extract, clove extract, ginseng extract, horse chestnut extract, witch hazel extract, and mulberry extract.

<Cosmetic Composition>

There is no particular limitation as to the method for producing the cosmetic composition according to the present invention and it can be appropriately selected for any purpose. For example, the cosmetic composition can be prepared by homogeneously mixing the above-described metal oxide particles, the above-described titanate particles, and, if necessary, other components, such as a dispersion medium.

The content of titanate particles in the cosmetic composition according to the present invention is preferably 0.1 to 200 parts by mass, more preferably 0.1 to 100 parts by mass, and still more preferably 1 to 40 parts by mass, relative to 100 parts by mass of metal oxide particles. By limiting the content of titanate particles within the above range, the titanate particles can further prevent the agglomeration of metal oxide particles and thus further increase the dispersibility of metal oxide particles in the cosmetic composition. Eventually, the capabilities that the metal oxide particles have can be sufficiently exerted.

There is no particular limitation as to the form of the cosmetic composition according to the present invention and it can be appropriately selected for any purpose. For example, the cosmetic composition can take a wide range of forms, such as gel, paste, oily liquid or emulsion without impairing the effects of the present invention. Specifically, the cosmetic composition can be widely applied in various forms, including powder, liquid, paste, lotion, cream, gel, and solid. Since in the cosmetic composition according to the present invention the dispersibility of metal oxide particles is increased, the cosmetic composition can be used, for example, for lotion, serum, essence emulsion, sunscreen lotion, sunscreen cream or foundation. A particularly preferred embodiment is a makeup cosmetic material into which titanium dioxide is required to be incorporated.

### EXAMPLES

The present invention will be described below in further detail with reference to specific examples. The present invention is not at all limited by the following examples and modifications and variations may be appropriately made therein without changing the gist of the invention.

### Production Example 1: Titanate Particles 1

Titanium dioxide, potassium carbonate, and lithium carbonate were weighed to give Ti:K:Li=1.73:0.8:0.27 (by molar ratio), these materials were mixed with further addition of potassium chloride as a flux to reach 55 parts by mass relative to 100 parts by mass of the total amount of titanium dioxide, potassium carbonate, and lithium carbonate, and the mixture was mixed for 10 minutes while being ground with a vibration mill. The obtained ground mixture was fired at 850° C. for four hours in an electric furnace and the fired product was ground to obtain a powder. The obtained powder was washed with water to remove potassium chloride and then dispersed into water, thus preparing a 20% by mass slurry. A 98% sulfuric acid was added to the slurry, followed by stirring for two hours to adjust the PH to 7. The solid content of the slurry was filtered out and dried at 110° C. After the drying, the dried product was fired at 600° C. for 12 hours in an electric furnace, thus obtaining a powder made of titanate particles 1.

The resultant powder was confirmed, with an inductively coupled plasma emission spectrometer (product number “SPS5100” manufactured by SII Nano Technology Inc.), to consist of lepidocrocite-type layered crystals of lithium potassium titanate (K_{0.7}Li_{0.27}Ti_{1.73}O_{3.95}). The shape of the resultant titanate particles 1 was platy.

### Production Example 2: Titanate Particles 2

Titanium dioxide, potassium carbonate, and lithium carbonate were weighed to give Ti:K:Li=1.73:0.8:0.27 (by molar ratio), these materials were mixed with further addition of potassium chloride as a flux to reach 55 parts by mass relative to 100 parts by mass of the total amount of titanium dioxide, potassium carbonate, and lithium carbonate, and the mixture was mixed for 10 minutes while being ground with a vibration mill. The obtained ground mixture was fired at 800° C. for four hours in an electric furnace and the fired product was ground to obtain a powder. The obtained powder was washed with water to remove potassium chloride and then dispersed into water, thus preparing a 20% by mass slurry. A 98% sulfuric acid was added to the slurry, followed by stirring for two hours to adjust the PH to 7. The solid content of the slurry was filtered out and dried at 110° C. After the drying, the dried product was fired at 600° C. for 12 hours in an electric furnace, thus obtaining a powder made of titanate particles 2.

The resultant powder was confirmed, with the same inductively coupled plasma emission spectrometer as in Production Example 1, to consist of lepidocrocite-type layered crystals of lithium potassium titanate (K_{0.7}Li_{0.27}Ti_{1.73}O_{3.95}). The shape of the resultant titanate particles 2 was platy.

### Production Example 3: Titanate Particles 3

Titanium dioxide, potassium carbonate, and lithium carbonate were weighed to give Ti:K:Li=1.73:0.8:0.27 (by molar ratio), these materials were mixed with further addition of potassium chloride as a flux to reach 20 parts by mass relative to 100 parts by mass of the total amount of titanium dioxide, potassium carbonate, and lithium carbonate, and the mixture was mixed for 10 minutes while being ground with a vibration mill. The obtained ground mixture was fired at 850° C. for four hours in an electric furnace and the fired product was ground to obtain a powder. The obtained powder was washed with water to remove potassium chloride and then dispersed into water, thus preparing a 20% by mass slurry. A 98% sulfuric acid was added to the slurry, followed by stirring for two hours to adjust the PH to 7. The solid content of the slurry was filtered out and dried at 110° C. After the drying, the dried product was fired at 600° C. for 12 hours in an electric furnace, thus obtaining a powder made of titanate particles 3.

The resultant powder was confirmed, with the same inductively coupled plasma emission spectrometer as in Production Example 1, to consist of lepidocrocite-type layered crystals of lithium potassium titanate (K_{0.7}Li_{0.27}Ti_{1.73}O_{3.95}). The shape of the resultant titanate particles 3 was platy.

### Production Example 4: Titanate Particles 4

Titanium dioxide, potassium carbonate, and lithium carbonate were weighed to give Ti:K:Li=1.73:0.8:0.27 (by molar ratio), these materials were mixed with further addition of potassium chloride as a flux to reach 20 parts by mass relative to 100 parts by mass of the total amount of titanium dioxide, potassium carbonate, and lithium carbonate, and the mixture was mixed for 10 minutes while being ground with a vibration mill. The obtained ground mixture was fired at 800° C. for four hours in an electric furnace and the fired product was ground to obtain a powder. The obtained powder was washed with water to remove potassium chloride and then dispersed into water, thus preparing a 20% by mass slurry. A 98% sulfuric acid was added to the slurry, followed by stirring for two hours to adjust the PH to 7. The solid content of the slurry was filtered out and dried at 110° C. After the drying, the dried product was fired at 600° C. for 12 hours in an electric furnace, thus obtaining a powder made of titanate particles 4.

The resultant powder was confirmed, with the same inductively coupled plasma emission spectrometer as in Production Example 1, to consist of lepidocrocite-type layered crystals of lithium potassium titanate (K_{0.7}Li_{0.27}Ti_{1.73}O_{3.95}). The shape of the resultant titanate particles 4 was platy.

### Production Example 5: Titanate Particles 5

Titanium dioxide, potassium carbonate, and lithium carbonate were weighed to give Ti:K:Li=1.73:0.8:0.27 (by molar ratio), these materials were mixed, and the mixture was mixed for 10 minutes while being ground with a vibration mill. The obtained ground mixture was fired at 850° C. for four hours in an electric furnace and the fired product was ground to obtain a powder. The obtained powder was dispersed into water, thus preparing a 20% by mass slurry. A 98% sulfuric acid was added to the slurry, followed by stirring for two hours to adjust the PH to 7. The solid content of the slurry was filtered out and dried at 110° C. After the drying, the dried product was fired at 600° C. for 12 hours in an electric furnace, thus obtaining a powder made of titanate particles 5.

The resultant powder was confirmed, with the same inductively coupled plasma emission spectrometer as in Production Example 1, to consist of lepidocrocite-type layered crystals of lithium potassium titanate (K_{0.7}Li_{0.27}Ti_{1.73}O_{3.95}). The shape of the resultant titanate particles 5 was platy.

### Production Example 6: Titanate Particles 6

Titanium dioxide, potassium carbonate, and lithium carbonate were weighed to give Ti:K:Li=1.73:0.8:0.27 (by molar ratio), these materials were mixed, and the mixture was mixed for 10 minutes while being ground with a vibration mill. The obtained ground mixture was fired at 950° C. for four hours in an electric furnace and the fired product was ground to obtain a powder. The obtained powder was dispersed into water, thus preparing a 20% by mass slurry. A 98% sulfuric acid was added to the slurry, followed by stirring for two hours to adjust the PH to 7. The solid content of the slurry was filtered out and dried at 110° C. After the drying, the dried product was fired at 600° C. for 12 hours in an electric furnace, thus obtaining a powder made of titanate particles 6.

The resultant powder was confirmed, with the same inductively coupled plasma emission spectrometer as in Production Example 1, to consist of lepidocrocite-type layered crystals of lithium potassium titanate (K_{0.7}Li_{0.27}Ti_{1.73}O_{3.95}). The shape of the resultant titanate particles 6 was platy.

### Production Example 7: Titanate Particles 7

Titanium dioxide, potassium carbonate, and lithium carbonate were weighed to give Ti:K:Li=1.73:0.8:0.27 (by molar ratio), these materials were mixed with further addition of potassium chloride as a flux to reach 20 parts by mass relative to 100 parts by mass of the total amount of titanium dioxide, potassium carbonate, and lithium carbonate, and the mixture was mixed for 10 minutes while being ground with a vibration mill. The obtained ground mixture was fired at 1200° C. for four hours in an electric furnace and the fired product was ground to obtain a powder. The obtained powder was washed with water to remove potassium chloride and then dispersed into water, thus preparing a 20% by mass slurry. A 98% sulfuric acid was added to the slurry, followed by stirring for two hours to adjust the PH to 7. The solid content of the slurry was filtered out and dried at 110° C. After the drying, the dried product was fired at 600° C. for 12 hours in an electric furnace, thus obtaining a powder made of titanate particles 7.

The resultant powder was confirmed, with the same inductively coupled plasma emission spectrometer as in Production Example 1, to consist of lepidocrocite-type layered crystals of lithium potassium titanate (K_{0.7}Li_{0.27}Ti_{1.73}O_{3.95}). The shape of the resultant titanate particles 7 was platy.

### Production Example 8: Titanate Particles 8

Titanium dioxide, potassium carbonate, and magnesium hydroxide were weighed to give Ti:K:Mg=1.6:0.8:0.4 (by molar ratio), these materials were mixed with further addition of potassium chloride as a flux to reach 55 parts by mass relative to 100 parts by mass of the total amount of titanium dioxide, potassium carbonate, and magnesium hydroxide, and the mixture was mixed for 10 minutes while being ground with a vibration mill. The obtained ground mixture was fired at 1150° C. for four hours in an electric furnace and the fired product was ground to obtain a powder. The obtained powder was washed with water to remove potassium chloride and then dispersed into water, thus preparing a 20% by mass slurry. A 98% sulfuric acid was added to the slurry, followed by stirring for two hours to adjust the PH to 7. The solid content of the slurry was filtered out and dried at 110° C. After the drying, the dried product was fired at 600° C. for 12 hours in an electric furnace, thus obtaining a powder made of titanate particles 8.

The resultant powder was confirmed, with the same inductively coupled plasma emission spectrometer as in Production Example 1, to consist of lepidocrocite-type layered crystals of magnesium potassium titanate (K_{0.7}Mg_{0.4}Ti_{1.6}O_{3.95}). The shape of the resultant titanate particles 8 was platy.

### Production Example 9: Titanate Particles 9

Titanium dioxide, potassium carbonate, lithium carbonate, and magnesium hydroxide were weighed to give Ti:K:Li:Mg=1.66:0.8:0.14:0.2 (by molar ratio), these materials were mixed with further addition of potassium chloride as a flux to reach 55 parts by mass relative to 100 parts by mass of the total amount of titanium dioxide, potassium carbonate, lithium carbonate, and magnesium hydroxide, and the mixture was mixed for 10 minutes while being ground with a vibration mill. The obtained ground mixture was fired at 1050° C. for four hours in an electric furnace and the fired product was ground to obtain a powder. The obtained powder was washed with water to remove potassium chloride and then dispersed into water, thus preparing a 20% by mass slurry. A 98% sulfuric acid was added to the slurry, followed by stirring for two hours to adjust the PH to 7. The solid content of the slurry was filtered out and dried at 110° C. After the drying, the dried product was fired at 600° C. for 12 hours in an electric furnace, thus obtaining a powder made of titanate particles 9.

The resultant powder was confirmed, with the same inductively coupled plasma emission spectrometer as in Production Example 1, to consist of lepidocrocite-type layered crystals of magnesium potassium titanate (K_{0.7}Li_{0.14}Mg_{0.2}Ti_{0.66}O_{3.95}) The shape of the resultant titanate particles 9 was platy.

Table 1 below shows the average unrolled diameters, average lengths, average unrolled diameter ratios, and average thicknesses of the resultant titanate particles 1 to 9 and commercially available mica, talc, and sericite used for cosmetics.

The shapes of the titanate particles 1 to 9, mica, talc, and sericite (particles) were observed with a scanning electron microscope (SEM, product number “S-4800” manufactured by Hitachi High-Technologies Corporation). More specifically, any 50 particles were selected for each kind of particles and their lengths and breadths were measured. The average unrolled diameter was obtained from the arithmetic average of the values of ((length)+(breadth))/2 of the 50 particles. The average length was obtained from the arithmetic average of the lengths of the 50 particles. The average unrolled diameter ratio was obtained from the arithmetic average of the values of (length)/(breadth) of the 50 particles. The average thickness was obtained by selecting any 10 particles by SEM observation, measuring their thicknesses, and taking the arithmetic average of the thicknesses of the 10 particles.

Each kind of particles, i.e., the resultant titanate particles 1 to 9 and commercially available mica, talc, and sericite used for cosmetics, were formed into a powder compact, the powder compact as a sample was measured in terms of reflectance, with a multi-angle spectrophotometer (product number “MA68II” manufactured by X-Rite, Inc.), by reflecting light at various angle onto the sample, and the angular dependency of the reflectance was calculated based on the formula below. The results are shown in Table 1. A smaller angular dependency indicates stronger reflectivity, a larger angular dependency indicates less shadow, and samples having an angular dependency of 80.0 to 93.0% take on a pretty white color without changing much in gloss and luster even when viewed from different angles by visual inspection.

Angular dependency [%]=[(reflectance at 1100)/(reflectance at 150)]×100

TABLE 1

| | | Average Unrolled | Average | Average Unrolled | Average | Angular |
|---|---|---|---|---|---|---|
| | | Diameter [μm] | Length [μm] | Diameter Ratio | Thickness [μm] | Dependency [%] |
| Titanate Particles 1 | K_{0.7}Li_{0.27}Ti_{1.73}O_{3.95} | 2.46 | 3.50 | 2.46 | 0.24 | 87.2 |
| Titanate Particles 2 | K_{0.7}Li_{0.27}Ti_{1.73}O_{3.95} | 0.83 | 1.10 | 2.17 | 0.15 | 92.8 |
| Titanate Particles 3 | K_{0.7}Li_{0.27}Ti_{1.73}O_{3.95} | 2.13 | 2.80 | 1.93 | 0.50 | 95.9 |
| Titanate Particles 4 | K_{0.7}Li_{0.27}Ti_{1.73}O_{3.95} | 1.07 | 1.30 | 1.68 | 0.27 | 96.7 |
| Titanate Particles 5 | K_{0.7}Li_{0.27}Ti_{1.73}O_{3.95} | 2.83 | 3.60 | 1.76 | 1.40 | 93.2 |
| Titanate Particles 6 | K_{0.7}Li_{0.27}Ti_{1.73}O_{3.95} | 8.81 | 11.00 | 1.67 | 3.00 | 91.2 |
| Titanate Particles 7 | K_{0.7}Li_{0.27}Ti_{1.73}O_{3.95} | 32.60 | 44.80 | 2.20 | 4.75 | 82.2 |
| Titanate Particles 8 | K_{0.7}Mg_{0.4}Ti_{1.6}O_{3.95} | 7.13 | 9.90 | 1.70 | 2.30 | 91.5 |
| Titanate Particles 9 | K_{0.7}Li_{0.14}Mg_{0.2}Ti_{1.66}O_{3.95} | 6.24 | 9.60 | 1.64 | 1.87 | 92.1 |

| | | | | | |
|---|---|---|---|---|---|
| Mica | 25.50 | 31.40 | 1.60 | 1.80 | 63.2 |
| Talc | 19.90 | 27.40 | 2.20 | 1.00 | 74.5 |
| Sericite | 6.40 | 8.10 | 1.70 | 0.50 | 75.6 |

### Examples 1 to 8, Comparative Examples 1 to 4

The titanate particles 1 to 9, mica, talc, sericite (test powder), and titanium dioxide (average particle diameter: 0.05 m, trade name “TTO-80 (A)” manufactured by Ishihara Sangyo Kaisha, Ltd.) were weight out to give each of mixed compositions shown in Table 2. The total amount of each mixture was 5 g. The mixture was put into a container and mixed for 10 seconds with a spoon. Thereafter, the mixed powder was formed into a powder compact. The average particle diameter of titanium dioxide was obtained by measuring the median diameter of its primary particles with a scanning electron microscope (product number “S-4800” manufactured by Hitachi High-Technologies Corporation).

The dispersibility of the mixed powder was evaluated as follows.

A powder compact of titanium dioxide alone and a powder compact of the test powder alone were measured in terms of value L with a colorimeter (product number “CR-300” manufactured by Konica Minolta, Inc.) and a value L under homogeneous dispersion (a calculated value L) was calculated based on the following formula.

Calculated value L=(value L of titanium dioxide alone)/2+(value L of test powder alone)/2

Furthermore, the resultant powder compact of each of the mixed powders was measured at any nine points in terms of value L (measured value L) with a colorimeter (product number “CR-300” manufactured by Konica Minolta, Inc.) and the deviation of the measured value L from the calculated value L was calculated using the standard deviation. Powder compacts having a standard deviation of less than 0.05 were evaluated as having a dispersibility indicated by “circle”, powder compacts having a standard deviation of not less than 0.05 and less than 0.10 were evaluated as having a dispersibility indicated by “triangle”, and powder compacts having a standard deviation of not less than 0.10 were evaluated as having a dispersibility indicated by “cross”. The results are shown in Table 2. As shown in Table 2, it can be seen that Examples 1 to 6 with the use of the titanate particles 1 to 6, respectively, and Examples 7 and 8 with the use of the titanate particles 8 and 9, respectively, were improved in the dispersibility of titanium dioxide as compared to platy minerals, such as mica and talc.

TABLE 2

| | | | | | | | | | | Comparative | | | |
|---|---|---|---|---|---|---|---|---|---|---|---|---|---|
| | | Examples | | | | | | | | Examples | | | |
| | | 1 | 2 | 3 | 4 | 5 | 6 | 7 | 8 | 1 | 2 | 3 | 4 |
| Mixed Composition [% by mass] | Titanium Dioxide | 50 | 50 | 50 | 50 | 50 | 50 | 50 | 50 | 50 | 50 | 50 | 50 |
| | Titanate Particles 1 | 50 | | | | | | | | | | | |
| | Titanate Particles 2 | | 50 | | | | | | | | | | |
| | Titanate Particles 3 | | | 50 | | | | | | | | | |
| | Titanate Particles 4 | | | | 50 | | | | | | | | |
| | Titanate Particles 5 | | | | | 50 | | | | | | | |
| | Titanate Particles 6 | | | | | | 50 | | | | | | |
| | Titanate Particles 7 | | | | | | | | | 50 | | | |
| | Titanate Particles 8 | | | | | | | 50 | | | | | |
| | Titanate Particles 9 | | | | | | | | 50 | | | | |
| | Mica | | | | | | | | | | 50 | | |
| | Talc | | | | | | | | | | | 50 | |
| | Sericite | | | | | | | | | | | | 50 |

| | | | | | | | | | | | | |
|---|---|---|---|---|---|---|---|---|---|---|---|---|
| Dispersibility | ◯ | ◯ | ◯ | ◯ | ◯ | Δ | ◯ | ◯ | X | X | X | X |

### Example 9 and Comparative Examples 5 to 8

Titanium dioxide (average particle diameter: 0.25 μm, trade name “CR-50” manufactured by Ishihara Sangyo Kaisha, Ltd.), the titanate particles 1, and mica were weight out to give each of mixed compositions shown in Table 3. The total amount of each mixture was 5 g. An amount of 20 g of acrylic resin was added into the mixture, followed by stirring at 2500 rpm for five minutes in a homo mixer. The obtained mixed resin was applied with a thickness of 200 μm onto a hiding chart and cured at 85° C. for 10 minutes. The average particle diameter of titanium dioxide was obtained by measuring the median diameter of its primary particles with a scanning electron microscope (product number “S-4800” manufactured by Hitachi High-Technologies Corporation).

The obtained hiding chart was measured at any three points of each of white and black portions in terms of value L with a colorimeter (product number “CR-300” manufactured by Konica Minolta, Inc.). A smaller lightness difference between the white and black portions indicates a more excellent hiding power. Therefore, the hiding power [%] was calculated based on the formula below using the difference in value L between white and black portions (lightness difference). The results are shown in Table 3. As shown in Table 3, it can be seen that the addition of the titanate particles 1 to titanium dioxide provided improvement in dispersibility, so that the hideability (hiding power) was increased as compared to titanium dioxide alone.

Hiding power [%]=[(100−(lightness difference))/100]×100

TABLE 3

| | | | Comp. | Comp. | Comp. | Comp. |
|---|---|---|---|---|---|---|
| | | Ex. 9 | Ex. 5 | Ex. 6 | Ex. 7 | Ex. 8 |
| Mixed | Titanium | 80 | 80 | 100 | 0 | 0 |
| Composition | Dioxide | | | | | |
| [% by mass] | Titanate | 20 | 0 | 0 | 100 | 0 |
| | Particles 1 | | | | | |
| | Mica | 0 | 20 | 0 | 0 | 100 |

| | | | | | |
|---|---|---|---|---|---|
| Hiding Power [%] | 99.8 | 96.9 | 99.1 | 83.8 | 40.3 |

### Example 10 and Comparative Example 9

The titanate particle 1, silicone-treated titanium dioxide (average particle diameter: 0.25 μm), silicone-treated yellow iron oxide (average particle diameter: 0.2 μm), silicone-treated red iron oxide (average particle diameter: 0.2 m), silicone-treated black iron oxide (average particle diameter: 0.3 μm), silicone-treated talc, silicone-treated mica, spherical silicone powder, methylphenylpolysiloxane, dimethylpolysiloxane, diisostearyl malate, petrolatum, and sorbitan monooleate were weight out to give each of mixed compositions (powdery foundations) shown in Table 4, and the mixture was stirred for five minutes with a Henschel mixer, and then pressed into a shape, thus obtaining a sample for a cosmetic composition. The average particle diameters of titanium dioxide and iron oxides were each obtained by measuring the median diameter of its primary particles with a scanning electron microscope (product number “S-4800” manufactured by Hitachi High-Technologies Corporation). Note that in Comparative Example 9 the titanate particles 1 were not used.

TABLE 4

| | | | Comp. |
|---|---|---|---|
| | | Ex. 10 | Ex. 9 |
| Mixed | Titanate Particles 1 | 15 | 0 |
| Composition | Silicone-Treated Titanium Dioxide | 10 | 10 |
| [% by mass] | Silicone-Treated Yellow Iron Oxide | 3.8 | 3.8 |
| | Silicone-Treated Red Iron Oxide | 2 | 2 |
| | Silicone-Treated Black Iron Oxide | 0.2 | 0.2 |
| | Silicone-Treated Talc | 23 | 38 |
| | Silicone-Treated Mica | 23 | 23 |
| | Spherical Silicone Powder | 10 | 10 |
| | Methylphenylpolysiloxane | 3 | 3 |
| | Dimethylpolysiloxane | 3 | 3 |
| | Diisostearyl Malate | 3 | 3 |
| | Petrolatum | 3 | 3 |
| | Sorbitan Monooleate | 1 | 1 |

### Example 11 and Comparative Example 10

The titanate particle 8, silicone-treated talc, cyclopentasiloxane, deionized water, silicone-treated titanium dioxide (average particle diameter: 0.25 μm), silicone-treated yellow iron oxide (average particle diameter: 0.2 μm), silicone-treated red iron oxide (average particle diameter: 0.2 m), silicone-treated black iron oxide (average particle diameter: 0.3 μm), dimethylpolysiloxane, and glycerin were weight out to give each of mixed compositions (liquid foundations) shown in Table 5, and the mixture was stirred for five minutes with a homo mixer, thus obtaining a sample for a cosmetic composition. The average particle diameters of titanium dioxide and iron oxides were each obtained by measuring the median diameter of its primary particles with a scanning electron microscope (product number “S-4800” manufactured by Hitachi High-Technologies Corporation). Note that in Comparative Example 10 the titanate particles 8 were not used.

TABLE 5

| | | | Comp. |
|---|---|---|---|
| | | Ex. 11 | Ex. 10 |
| Mixed | Titanate Particles 8 | 5 | 0 |
| Composition | Silicone-Treated Talc | 5 | 10 |
| [% by mass] | Cyclopentasiloxane | 30 | 30 |
| | Deionized Water | 40 | 40 |
| | Silicone-Treated Titanium Dioxide | 8 | 8 |
| | Silicone-Treated Yellow Iron Oxide | 3 | 3 |
| | Silicone-Treated Red Iron Oxide | 1.5 | 1.5 |
| | Silicone-Treated Black Iron Oxide | 0.5 | 0.5 |
| | Dimethylpolysiloxane | 4 | 4 |
| | Glycerin | 3 | 3 |

### Example 12 and Comparative Example 11

The titanate particle 9, silicone-treated talc, silicone-treated mica, silicone-treated titanium dioxide (average particle diameter: 0.25 μm), silicone-treated zinc oxide (average particle diameter: 0.4 μm), dimethylpolysiloxane, and 1,3-butylene glycol were weight out to give each of mixed compositions (loose powders) shown in Table 6, and the mixture was stirred for five minutes with a Henschelmixer, thus obtaining a sample for a cosmetic composition. The average particle diameters of titanium dioxide and zinc oxide were each obtained by measuring the median diameter of its primary particles with a scanning electron microscope (product number “S-4800” manufactured by Hitachi High-Technologies Corporation). Note that in Comparative Example 11 the titanate particles 9 were not used.

TABLE 6

| | | | Comp. |
|---|---|---|---|
| | | Ex. 12 | Ex. 11 |
| Mixed | Titanate Particles 9 | 10 | 0 |
| Composition | Silicone-Treated Talc | 50 | 60 |
| [% by mass] | Silicone-Treated Mica | 20 | 20 |
| | Silicone-Treated Titanium Dioxide | 7 | 7 |
| | Silicone-Treated Zinc Oxide | 3 | 3 |
| | Dimethylpolysiloxane | 5 | 5 |
| | 1,3-Butylene Glycol | 5 | 5 |

By applying the cosmetic compositions obtained in Examples 10 to 12 and Comparative Examples 9 to 11 onto skin and visually observing them, the incorporation of lepidocrocite-type platy titanate particles was confirmed to bring about the effect of reducing color unevenness and increasing hideability as compared to the case where lepidocrocite-type platy titanate particles were not incorporated.

1. A cosmetic composition containing metal oxide particles with an average particle diameter of 1 μm or less and lepidocrocite-type platy titanate particles with an average unrolled diameter of 0.1 μm to 10.0 μm and an average thickness of 0.1 μm to 4.0 μm,
the titanate particles being at least one selected from titanates expressed by chemical formulae K_{0.5-0.7}Li_{0.27}Ti_{1.73}O_{3.85-3.95}, K_{0.2-0.7}Mg_{0.4}Ti_{1.6}O_{3.7-3.95}, and K_{0.2-0.7}Li_{0.27-(2x/3)}MgₓTi_{1.73-(x/3)}O_{3.7-3.95} [where 0.004≤x≤0.4].

2. The cosmetic composition according to claim 1, wherein the metal oxide particles are at least one selected from the group consisting of titanium dioxide, zinc oxide, iron oxide, aluminum oxide, cerium oxide, zirconium oxide, silicon oxide, chromium oxide, magnesium oxide, and black titanium oxide.

3. The cosmetic composition according to claim 1, wherein the metal oxide particles have an average particle diameter of 0.01 μm to 0.5 μm.

4. The cosmetic composition according to claim 1, wherein the titanate particles have an average length of less than 10 μm.

5. The cosmetic composition according to claim 1, wherein the titanate particles have an average unrolled diameter ratio of 1 to 5.

6. The cosmetic composition according to claim 1, wherein a content of the titanate particles is 0.1 to 200 parts by mass relative to 100 parts by mass of the metal oxide particles.
